(19) United States
(12) Patent Application Publication
(10) Pub. No.: US 2012/0009134 A1
(43) Pub. Date: Jan. 12, 2012

(54) Bleaching Agent Having Delayed Bleaching Action

(76) Inventors: Carolin Welz, Hamburg (DE); Hartmut Manneck, Klein Wesenberg (DE); Astrid Kleen, Hamburg (DE); Mustafa Akram, Hamburg (DE)

(21) Appl. No.: 13/234,254

(22) Filed: Sep. 16, 2011

Related U.S. Application Data
(63) Continuation of application No. PCT/EP2010/052660, filed on Mar. 3, 2010.

(30) Foreign Application Priority Data
Mar. 18, 2009 (DE) 10 2009 001 636.8

(51) Int. Cl. A61K 8/84; B05D 1/36; B05D 7/00; A61K 8/11; A61Q 5/08

(52) U.S. Cl. 424/62; 424/401; 427/214

(57) ABSTRACT

Agents for lightening and/or bleaching keratin-containing fibers, particularly human hair, comprising coated bleaching and/or alkalizing agents. The agent provides uniform lightening regardless of which strands are treated first or last with the hair-bleaching agent. The present invention further relates to a method for producing lightening and/or hair-bleaching agents for keratinic fibers containing coated particles.

### CROSS REFERENCE TO RELATED APPLICATIONS

This application is a continuation of International Patent Application No. PCT/EP2010/052660 filed 3 Mar. 2010, which claims priority to German Patent Application No. 10 2009 001 636.8 filed 18 Mar. 2009, both of which are incorporated herein by reference.

The invention relates to agents for lightening and/or bleaching keratin-containing fibers, particularly human hair, which contain encased bleaching and/or alkalizing agents. The intention thereby is to provide uniform lightening regardless of which strands are treated first or last with the hair-bleaching agent. The formulation according to the present invention allows the user, despite time-intensive strand-by-strand application, to rinse out all the strands simultaneously at the end of the hair-bleaching process, since the hair has been uniformly bleached due to the delayed start of the bleaching process. The delay in bleaching is achieved by encasing and/or encapsulating individual, or all, bleaching agent constituents. Shellac has shown itself in this context to be a particularly preferred encapsulation material. A further subject of the present invention relates to a method for manufacturing lightening and/or hair-bleaching agents for keratinic fibers containing coated particles.

Modification of the shape and color of hair represents an important sector of modem cosmetology. The appearance of the hair can thereby be adapted both to current fashion trends and to the individual desires of the particular person. So-called hair-bleaching, bleaching or lightening agents, which are used synonymously hereinafter, are especially popular for use by consumers.

Commercial high-performance bleaching agents are typically hair-bleaching powders made up of one or more persulfate salts, sodium silicate (water glass) and adjusting agents, which are mixed with a hydrogen peroxide solution immediately before use. Bleaching agents of this kind usually present the consumer with the problem of inhomogeneous coloring. In order to achieve homogeneous color results, it is particularly important to control the starting point of the coloring process. This problem is especially severe when many selected hair areas, distributed over the head, need to be colored. Coloring agents are often used to lighten only individual hair strands in order to produce a visually interesting hair color. In conventional “highlighting” processes, individual hair strands are placed into pieces of foil, treated with the hair-bleaching preparation, and wrapped in the foil. Because this is a time-consuming application process, the contact times on the successively treated strands are different, so that some of the hair treated is already sufficiently bleached while in other portions the bleaching process has not yet begun. This results in strands bleached to different intensities that, because they are different, are undesirable. This necessitates rinsing the hair out partially during the hair-bleaching process. Greater time independence when applying and rinsing out the hair-bleaching agents is therefore desirable for the user, especially the professional user.

There has therefore been no lack of attempts to develop suitable methods for delaying the onset of coloring.

One approach is represented by selective encapsulation of ingredients. DE 199 43 925 A1 discloses a bleaching agent coated with C₁₂ to C₂₄ fatty acid sugar esters. DE 10 2004 038 991 A1 discloses a bleaching agent containing an encased alkalizing agent, the casing being made up at a proportion of at least 50 wt % of complexing agents. DE 10 2005 038 073 A1 discloses bleaching agents for keratin-containing fibers having a coated alkalizing agent encompassing a particle core that contains alkalizing agent, and comprises a casing that surrounds the core and is made up of fatty acids, lactones, acid anhydrides, and/or water-soluble polymers. DE 10 2007 041 492 A1 likewise describes hair-bleaching agents with a delayed bleaching onset containing a solid alkalizing agent, the alkalizing agent encompassing a particle core containing the alkalizing agent and a casing surrounding the core and is made up of specific water-insoluble organic oils and waxes. WO 1997 007776 A1 discloses dust-free hair bleaching agents containing peroxo compounds encased with alkylated diesters and cyclic ethers and esters. The encasing of pH adjusting agents in hair coloring agents is known from EP 1 752 191 A2, which discloses encased alkalizing agents, and from WO 89 065531 A1, which describes encapsulated acidifying agents.

The present invention therefore is directed towards agents for lightening keratin-containing fibers that provide a uniform and homogeneous lightening result, particularly with the “highlighting” technique. The intent is to avoid over-bleaching of individual strands that are acted upon by the highlighting mixture earlier in the process than other strands.

It has now been found that the aforesaid objects can be achieved by hair-bleaching agents comprising at least one particulate having a particle core and a coating made up of specific encasing substances. In contrast to usual hair-bleaching agents, with the hair-bleaching agent according to the present invention at least some or all constituents are encapsulated or coated in order to release the active substances in delayed fashion. Organic and inorganic materials that are poorly soluble in the aqueous developer preparation and that act as a delay switch are appropriate for the coating. Shellac has proven to be particularly advantageous as an encasing or encapsulation material with regard to delayed active-substance release.

A first subject of the present invention is therefore coated particles having a solid particle core and contain a casing surrounding the core, wherein the casing contains at least shellac.

Shellac is obtainable from the secretion of lac insects (Kerria lacca resp. Laccifer lacca), and occurs as a stiff resin having an average molecular weight of approx. 1000 g/mol. It is made up predominantly of partly unsaturated hydroxycarboxylic acids that are present either as esters with one another or as a lactone. The principal components are aleuritic acid (aleuric acid, 9,10,16-trihydroxypalmitic acid) with up to approx. 32 wt % and shellolic acid.

Shellac is readily soluble in alcohols, organic acids, and aqueous bases, less so in esters and ketones, and insoluble in hydrocarbons and water.

The recovery of shellac involves collecting the secretion deposited onto branches, removing twig residues, and decolorizing with alkali. From this “seed lac,” the actual shellac is isolated as a wax-containing or wax-free resin. The shellac (commercial names: Lemon, TN, Ivory, Orange, Honey), as produced after a melt filtration process in which melted seed lac is filtered in order to remove accompanying substances, still contains the natural wax proportion of approx. 4 to 6 wt %. Bleached shellac occurs as a white powder when chlorine bleach liquor (sodium hypochlorite) acts on seed lac; it is available as both wax-containing and wax-free. Wax-free shellac, which is obtained from seed lac by solvent extraction accompanied by (partial) decolorizing using activated carbon and is present after drying in the form of thin flakes, is marketed as “flake shellac”. Wax-free shellac is marketed, for example, under the trade names Shellac SSB 55 Astra FL (wax content max. 0.2 wt %), Shellac CZSH 2 (wax content max. 0.25 wt %), Mantrolac R 49 (wax content max. 0.2 wt %), Aquagold (approx. 25 wt % aqueous solution with pH 7.3, SSB Co.), and Angelo Dewaxed Garnet Shellac.

Wax-free or low-wax shellac is used particularly preferably according to the present invention as an encapsulation material. “Low-wax” shellac is understood here as shellac having a weight proportion of wax of at most 0.5 wt %.

Because of its large number of functional groups, shellac is easy to harden and chemically modify. In particular, it is outstandingly easy to mix with further encasing agents.

It has been found that it is particularly advantageous according to the present invention if the casing material also contains at least one further component. Certain clay minerals, especially sheet silicates, have proven to be particularly advantageous as further components. Sheet silicates usable according to the present invention are serpentine, kaolinite, and particularly phyllosilicates such as pyrophyllite and, very particularly preferably, talc.

Talc or talcum (INCI name: Talc) is an insoluble magnesium silicate (Mg₃(OH)₂/[Si₄O₁₀] or 3MgO.4SiO₂.H₂O) and is used, usually as a white powder, as a filler material in casings in the pharmaceutical industry and the food industry.

A preferred embodiment of the present invention is therefore coated particles having a solid particle core and containing a casing surrounding the core, wherein the casing is made up of at least two components, the two components being shellac and talc.

Coated particles whose casing is made up of the two components shellac and talc at a proportion of at least 50 wt %, preferably at least 60 wt %, particularly preferably at least 70 wt %, based on total weight of the casing, are particularly preferred.

In order to ensure a delay in the release of the particle cores into the hair-bleaching preparation, it has been found that the two components shellac and talc should be present at a specific weight ratio to one another. A preferred embodiment of the present invention is therefore coated particles wherein the two components shellac and talc in the casing are present at a weight ratio to one another of from 5:1 to 1:1, preferably 3:1 to 3:2.

A casing material usable with particular advantage according to the present invention is the raw material marketed by the Biogrund company under the trade name Bonulac, which is made up of shellac (50 to 60%), ammonium hydrogencarbonate (5 to 10%), fractionated coconut oil (2 to 5%), glycerol (3 to 7%), and talc (20 to 30%).

All solid constituents of a hair-bleaching preparation are, in principle, usable as a particle core. Preferably, all solid constituents of the hair-bleaching preparation are covered with the encasing material. In a further embodiment of the present invention, only some of the constituents are used, or specific substance classes that can be functionally delimited, as a particle core in the hair-bleaching agent. Included among these functional substance classes are, in particular, those that have a direct influence on the onset of the hair-bleaching process, such as peroxo compounds or alkalizing agents. Because these compounds are coated, the onset of the actual lightening operation is delayed and greater time independence in application of the hair-bleaching agent is thus achieved for the user. Uniform and gentle lightening is therefore obtained, even in the event of poor timing on the part of the user when rinsing the strands.

Solid peroxo compounds and solid alkalizing agents are therefore preferably suitable as encased particle cores. Due to the encasing, the oxidizing agent in the form of the peroxo compound required for the bleaching effect, and/or the pH adjusting agent required for hair bleaching are thus released only in delayed fashion.

A preferred embodiment of the present invention is therefore one wherein the encased particles contain solid alkalizing agents and/or solid peroxo compounds as a particle core.

The solid alkalizing agent serves to establish an alkaline pH in the utilization mixture. According to the present invention, typical alkalizing agents known to one skilled in the art of hair-bleaching agents, such as ammonium hydroxides, alkali metal hydroxides, and alkaline-earth metal hydroxides, as well as the correspondingly analogous carbonates, silicates, in particular metasilicates, and alkali metal phosphates, can be used. Amino acids that react in alkaline fashion, such as arginine, ornithine, and lysine, are also usable.

In a preferred embodiment, solid alkalizing agents chosen from alkali metal carbonates, metasilicates, silicates, and phosphates, in particular alkali metal silicates and alkali metal metasilicates, are contained as a particle core.

In a further preferred embodiment, the particles contain as a particle core at least two different alkalizing agents. Mixtures, for example of a metasilicate and a carbonate, can be preferred in this context.

A further preferred embodiment of the present invention is encased particles containing peroxo compounds as a coated particle core.

Peroxo compounds known to one skilled in the art are usable according to the present invention, for example, hydrogen peroxide addition compounds on solid carriers, ammonium and alkali metal peroxomonosulfates and peroxodisulfates, alkali metal peroxodiphosphates such as potassium peroxodiphosphate, percarbonates such as magnesium percarbonate, peroxides such as barium peroxide, and perborates such as sodium perborate. Particularly preferred peroxo compounds are chosen from ammonium peroxodisulfate, potassium peroxodisulfate, sodium peroxodisulfate, ammonium peroxomonosulfate, potassium peroxomonosulfate, sodium peroxomonosulfate, as well as hydrogen peroxide addition compounds on solid carriers, urea peroxide, and melamine peroxide. Among these peroxo compounds, which can also be used in combination, the inorganic compounds are preferred according to the present invention. Peroxodi sulfates are particularly preferred, particularly combinations of at least two peroxodisulfates. The combination of ammonium peroxodisulfate and sodium peroxodisulfate and the combination of ammonium peroxodisulfate and potassium peroxodisulfate are particularly preferred in this context.

A further preferred embodiment of the present compound is that wherein the encased particles contain as a particle core solid peroxo compounds chosen from hydrogen peroxide addition compounds on solid carriers, ammonium peroxomonosulfates, alkali metal peroxomonosulfates, ammonium peroxodisulfates, and alkali metal peroxodisulfates.

It may be advantageous according to the present invention if the encased particles contain further, additional encasing materials in the casing material. Various encapsulating agents or casing materials can be combined with one another in order to achieve a specific time delay. The various casing materials can be mixed with one another before coating, and applied as a co-casing material onto the particle core. It may also be desirable, however, to influence release by way of different casing materials. It may be advantageous for that purpose to apply the various coating materials successively, so that the particle core is surrounded by casings that differ in terms of their properties.

Preferred additional casing materials in this context include derivatives of cellulose, particularly cellulose ethers. Such cellulose ethers are obtainable by reacting cellulose with alkylating agents such as alkyl halides or epoxides. Preferred alkylating agents are methyl chloride, ethyl chloride, 1-propyl chloride, 2-propyl chloride, methyl bromide, ethyl bromide, 1-propyl bromide, 2-propyl bromide, ethylene oxide, propylene oxide, 1-butylene oxide, 2-butylene oxide, and isobutylene oxide. When epoxides and alkyl halides are used simultaneously, substitution distributions of alkylations, hydroxyalkylations, and alkoxyalkylations occur. The latter are created as a result of hydroxyalkylation and subsequent alkylation on the hydroxy functionality just formed.

Additional casing materials particularly preferred according to the present invention are cellulose ethers chosen from compounds having the INCI names Hydroxypropyl Cellulose, Hydroxypropyl Methylcellulose, and Ethyl Cellulose. Coating materials of this kind are obtainable commercially and marketed, for example, under the trade names Methocel® E, Methocel® F, Methocel® J, Methocel® K, and Methocel® 310, as well as Ethocel® or Methocel® A by Dow Chemicals, or under the trade name Surelease® by the Colorcon company.

HPMC (hydroxypropylmethyl cellulose) and the casing material marketed under the trade name Aquapolish by the Biogrund company, which is made up of hydroxypropylmethyl cellulose (HPMC), hydroxypropyl cellulose (HPC), talc, stearic acid, and microcrystalline cellulose, have proven to be a particularly advantageous additional casing material.

If the casing is not made up at a proportion of 100% of the aforesaid substances, it can contain further ingredients such as plasticizers, disintegration agents, separating agents, dyes, and scents, or adjuvants.

Particularly preferred particles according to the present invention contain plasticizers in the casing, for better casing elasticity. Plasticizers derive preferably from the group constituted from dialkyl phthalates, in particular diethyl phthalate, triethyl citrate, glycerol triacetate, and/or the polyethylene glycols.

In a further embodiment, coated particles according to the present invention additionally contain at least one disintegration agent in the casing surrounding the particle core.

Disintegration agents of this kind are often also described in the literature as disintegrating agents or bursting agents. Substances of this kind are incorporated into the polymer casing in order to shorten its disintegration time and adjust it to a desired duration. A duration from 15 to 30 minutes is preferred in this context according to the present invention. This disintegration of the casing occurs in particular because of an increase in volume due to water penetration (swelling).

Cellulose-based disintegration agents are preferred disintegration agents in the context of the present invention. Suitable celluloses are made up of approximately 500 to 5000 glucose units, and consequently have average molar weights from 50,000 to 500,000. Also usable cellulose-based disintegration agents are cellulose derivatives obtainable from cellulose via polymer-analogous reactions. Such chemically modified celluloses include products of esterification or etherification processes in which hydroxy hydrogen atoms have been substituted. Celluloses in which the hydroxy groups have been replaced with functional groups that are not bound via an oxygen atom can also be used as cellulose derivatives. Cellulose derivatives include alkali celluloses, carboxymethyl cellulose (CMC), cellulose esters and ethers, and aminocelluloses. These cellulose derivatives are preferably not used as the only cellulose-based disintegration agent, but in a mixture with cellulose. The cellulose-derivative content of these mixtures is preferably below 50 wt %, more preferably below 20 wt %, based on the cellulose-based disintegration agent. Pure cellulose that is free of cellulose derivatives is particularly preferred for use as a cellulose-based disintegration agent. Microcrystalline cellulose can be used as a further cellulose-based disintegration agent or as a constituent of that component. This microcrystalline cellulose is obtained by partial hydrolysis of celluloses under conditions such that only the amorphous regions (approx. 30% of the total cellulose mass) of the celluloses are attacked and dissolve completely, but the crystalline regions (approx. 70%) remain undamaged. A subsequent disaggregation of the microfine celluloses resulting from hydrolysis yields the microcrystalline celluloses, which have primary particle sizes of approx. 5 μm and are compactable, for example, into granules having an average particle size of 200 μm. Suitable microcrystalline cellulose is commercially obtainable, for example, under the trade name Emcocel® from the JRS Pharma company, or Avicel® from the FMC company. Suitable carboxymethyl cellulose derivatives are marketed, for example, under the trade name Tylopur® by the Clariant company, or Ac-Di-Sol® by the FMC company.

In addition, starch can also be used as a disintegration agent. Starch usable according to the present invention is usually obtained from vegetable raw materials, such as rice, soybeans, potatoes, or corn. Starch can be unmodified or (by analogy with cellulose) modified starch. Particularly preferred starch modifications are furnished by esterification and etherification reactions, particularly the ethers obtained from reactions with hydroxycarboxylic acids. A starch modification that is particularly suitable according to the present invention is the mixture of sodium carboxymethyl starch and sodium glycol starch that is marketed by the JRS Pharma company under the trade name Explotab®. Disintegration agents based on corn starch are particularly preferred according to the present invention. Suitable modified corn starches are obtainable, for example, under the trade names Glycolys® from the Roquette company, or Starch 1500® from the Colorcon company.

Lastly, disintegration agents made up of crosslinked, water-insoluble polyvinylpyrrolidinone (PVP) represent a further class of disintegration agents that are particularly suitable according to the present invention. The advantageous crosslinking of this PVP modification is based principally on entanglements and loops of the individual polymer strands with one another. A PVP-based disintegration agent that is particularly preferred according to the present invention is marketed by the BASF company under the trade name Kollidon® CL.

Coatings according to the present invention that surround the particle core preferably contain the disintegration adjuvant in quantities from 0.05 to 20 wt %, more preferably from 0.1 to 10 wt %, based on total weight of the dried casing.

It is further preferred according to the present invention if the casing surrounding the particle core additionally contains at least one separating agent and/or at least one pore-forming agent.

In a preferred embodiment, the coated particles therefore additionally contain in the casing surrounding particle core at least one separating agent. The separating agent serves to prevent sticking or caking of the encased particles during the manufacturing process and/or during storage and/or, if applicable, during mixing with further preparations. Talc, amorphous silicon dioxide marketed, for example, under the trade name Syloid® 244FP by Grace GmbH, glycerol monostearate, magnesium stearate, and silicates have, in particular, proven successful as separating agents.

Preferred particles according to the present invention are characterized in that the particulate constituents contain silicon dioxide as an additional separating agent.

In a further embodiment, coated particles according to the present invention additionally contain in the casing surrounding the particle core at least one pore-forming agent. Pore-forming agents are additionally incorporated into the coating and cause pores to form in the surface of the coating. This results in an increase in the rate of diffusion of hydrophilic substances, particularly water, into the polymer casing. Suitable pore-forming agents include polyvinylpyrrolidinone, sugars and sugar alcohols such as lactose, sucrose, sorbitol, and mannitol, polyethylene glycols having fewer than 600 ethylene oxide units, as well as cellulose derivatives such as hydroxypropyl cellulose, hydroxypropylmethyl cellulose, methyl cellulose, and mixtures thereof. Particularly preferred pore-forming agents according to the present invention are polyvinylpyrrolidinones (PVP), marketed,for example, under the trade name Kollidon® by the BASF company. According to the present invention, the weight proportion of pore-forming agents in the dried casing is from 0.5 to 50 wt %, particularly from 1 to 30 wt %, based on total weight of the casing.

In a process according to the present invention for manufacturing the casing, the encapsulation agent as well as the further adjuvants as applicable are homogeneously mixed and sprayed as an aqueous dispersion by fluidized bed technology (using the bottom spray method with or without the Wurster process, or the tangential spray method), onto the powdered particle cores, particularly the solid alkalizing agents and solid peroxo compounds, and at the same time are dried in the air flow so that a uniform, almost 100% layer surrounds the powder particles.

As an alternative to fluidized bed technology, coatings with the aforesaid film materials are alternatively possible using spouted bed technology. A further possibility for encasing powder particles is melt extrusion technology.

It may be advantageous to encapsulate the particle cores with a multi-layer casing. Dispersions that are identical or that differ in terms of their composition regarding encapsulation materials, separating agents, and adjuvants can be applied successively, and optionally can also be applied using different coating techniques.

In a particular embodiment of the present invention, it has proven to be particularly advantageous if, for particularly effective elimination of sticking or caking of the particles, the particulate constituents contain silicon dioxide as an additional separating agent, in particular amorphous and/or pyrogenic silicon dioxide, marketed, for example, under the trade names Syloid 244FP by Grace GmbH, or Aerosil by the Evonik company.

For this purpose, the particulate components can be mixed directly with silicon dioxide, particularly as long as the particles have not yet completely dried. In a preferred embodiment, the particulate components containing silicon dioxide as an additional separating agent are manufactured by spraying the particulates, already coated with one or more casings, with an aqueous dispersion containing silicon dioxide, and then drying the sprayed particulates. A silicon dioxide-containing dispersion of this kind preferably contains from 5 to 30 wt % silicon dioxide and is marketed, for example, by the Evonik company under the trade name Aerodisp W.

In a further embodiment, subsets of the particle cores are encapsulated with different encasing or coating materials. In a further manufacturing process step, these partial batches are mixed with one another at specific proportions.

Agents according to the present invention contain coated particles having an average particle diameter from 5 to 1000 μm, preferably from 50 pm to 500 μm, and more preferably from 100 μm to 250 μm. The coating material is preferably applied in a specific quantity onto the particle cores to be coated. It is preferred that the casing account for 10 to 70 wt %, preferably 15 to 65 wt %, of the total weight of the coated particles.

The particles described are preferably used in cosmetic preparations in order to lighten hair.

A further subject of the present invention therefore relates to an agent for lightening keratinic fibers, particularly human hair, containing in a cosmetic carrier at least one oxidizing agent, preferably hydrogen peroxide, and further containing at least encased particles in accordance with the first subject of the invention.

Statements made regarding the particles according to the present invention apply, mutatis mutandis, to the coated particles contained in the agents according to the present invention. “Keratinic fibers” or “keratin fibers” refers to furs, wool, feathers, and in particular human hair.

Agents according to the present invention contain the oxidation agents and encased particles in a cosmetic carrier. This cosmetic carrier is, for purposes of the invention, aqueous, alcoholic, or aqueous alcoholic. For hair coloring such carriers include creams, emulsions, gels, or surfactant-containing foaming solutions such as shampoos, foam aerosols, or other preparations suitable for use on the hair. “Aqueous alcoholic” carriers refer to water-containing compositions containing 3 to 70 wt % of a C₁ to C₄ alcohol, based on total weight of the utilization mixture, particularly ethanol, propylene glycol, glycerol, or isopropanol. An aqueous carrier contains, for purposes of the invention, at least 30 wt %, in particular at least 50 wt %, water, based on total weight of the utilization mixture.

The amount of oxidizing agent in the ready-to-use agent is preferably 0.5 to 12 wt %, more preferably 2 to 10 wt %, particularly preferably 3 to 6 wt % (calculated as 100% H₂O₂), based on total weight of the ready-to-use agent.

Oxidizing agent preparations of this kind are preferably aqueous, flowable oxidizing agent preparations. Preferred preparations are those wherein the flowable oxidizing agent preparation contains 40 to 95 wt %, preferably 50 to 90 wt %, more preferably 55 to 85 wt %, particularly preferably 60 to 80 wt %, and in particular 65 to 75 wt % water, based on its weight.

According to the present invention the lightening agent can also be applied together with a catalyst onto the hair. Such catalysts include specific enzymes such as peroxidases, iodides, quinones, or metal ions such as Zn²⁺, Cu²⁺, Fe²⁺, Fe³⁺, Mn²⁺, Mn⁴⁺, Li⁺, Mg²⁺, Ca²⁺, Ca²⁺, Ce⁴⁺, V³⁺, Co²⁺, Ru³⁺and Al³⁺, particularly Zn²⁺, Cu²⁺ and Mn²⁺.

It has furthermore proven to be advantageous if the oxidizing agent preparations contain at least one stabilizer or complexing agent. Particularly preferred stabilizers are phenacetin, alkali benzoates (sodium benzoate) and salicylic acid. Use of complexing agents is also preferred according to the present invention. Complexing agents are substances that can complex metal ions. Preferred complexing agents are chelate complexing agents, that is, substances that form cyclic compounds with metal ions, a single ligand occupying more than one coordination site on a central atom (i.e., being at least “double-toothed”). All complexing agents in the art can be used in the context of the present invention. Preferred complexing agents are nitrogen-containing polycarboxylic acids, particularly EDTA, and phosphonates, preferably hydroxyalkane- or aminoalkanephosphonates and in particular 1-hydroxyethane-1,1-diphosphonate (HEDP), ethylenediaminetetramethylenephosphonate (EDTMP), diethylenetriaminepentamethylenephosphonate (DTPMP), as well as physiologically acceptable salts of those compounds.

Agents according to the present invention can also contain further adjuvants and additives. It is preferred if the coloring preparation and/or the oxidizing agent preparation contain at least one thickening agent. No limitations exist in principle with regard to these thickening agents. Organic polymers such as anionic or nonionic synthetic polymers, natural thickening agents such as biosaccharide gums, alginates, or starch, as well as entirely inorganic thickening agents such as bentonite, in particular smectites, can be utilized.

To further enhance the performance of the oxidizing agent preparation, at least one optionally hydrated SiO₂ compound can additionally be added to the composition. It may be preferred to use optionally hydrated SiO₂ compounds in amounts from 0.05 wt % to 15 wt %, more preferably in quantities from 0.15 wt % to 10 wt %, and particularly preferably in quantities from 0.2 wt % to 5 wt %, based on total weight of the composition according to the present invention. Quantitative indications reflect in each case the concentration of the SiO₂ compounds (without their water component) in the agents. Optionally hydrated SiO₂ compounds can be present in various forms. Preferably, SiO₂ compounds in the form of silica gels, or particularly preferably as water glass, are used. These SiO₂ compounds can be present in part in aqueous solution. Water glasses that are particularly preferred are marketed, inter alia, by the Henkel Company under the designations Ferrosil 119, Natronwasserglas 40/42, Portil A, Portil AW, and Portil W, and by the Akzo Company under the designation Britesil C20.

Agents according to the present invention are preferably packaged as flowable preparations. For that purpose, an emulsifier or surfactant is preferably further added to the agents, surface-active substances being referred to as surfactants or as emulsifiers depending on the field of application, and being chosen from anionic, cationic, zwitterionic, amphoteric, and nonionic surfactants and emulsifiers.

Preferred anionic surfactants are alkyl sulfates, alkyl ether sulfates, and ethercarboxylic acids having 10 to 18 carbon atoms in the alkyl group and up to 12 glycol ether groups in the molecule. Particularly suitable zwitterionic surfactants are betaines, such as the fatty acid amide derivative known by the INCI name Cocamidopropyl Betaine. Particularly preferred amphoteric surfactants are N-cocalkylaminopropionate, cocacylaminoethylaminopropionate, and C₁₂ to C₁₈ acyl sarcosine. Suitable nonionic surfactants include addition products of ethylene oxide and/or propylene oxide with linear and branched fatty alcohols having 8 to 30 carbon atoms, for example, lauryl, myristyl, cetyl, but also stearyl, isostearyl, and oleyl alcohol, as well as C₈ to C₂₂ alkyl mono- and oligoglycosides. Preparations having outstanding properties are likewise obtained when they contain, as nonionic surfactants, fatty acid esters of ethoxylated glycerol.

For surfactants that represent addition products of ethylene oxide and/or propylene oxide, products having a “normal” homolog distribution as well as those having a restricted homolog distribution can be used. A “normal” homolog distribution refers to mixtures of homologs obtained when reacting fatty alcohol and alkylene oxide using alkali metals, alkali metal hydroxides, or alkali metal alcoholates as catalysts. Restricted homolog distributions are obtained when, for example, hydrotalcites, alkaline-earth metal salts of ethercarboxylic acids, or alkaline-earth metal oxides, hydroxides, or alcoholates are used as catalysts. Use of products having a restricted homolog distribution can be preferred.

Anionic, nonionic, zwitterionic, or amphoteric surfactants are used in quantities from 0.1 to 45 wt %, preferably 1 to 30 wt %, and very particularly preferably from 1 to 15 wt %, based on total weight of the ready-to-use agent.

Likewise preferred according to the present invention are cationic surfactants of the quaternary ammonium compound, esterquat, and amidoamine types. Further cationic surfactants usable according to the present invention include quaternized protein hydrolysates. Cationic surfactants are present in agents according to the present invention preferably in quantities from 0.05 to 10 wt %, based on the entire agent. Quantities from 0.1 to 5 wt % are particularly preferred.

In a preferred embodiment, nonionic, zwitterionic, and/or amphoteric surfactants, as well as mixtures thereof, can be preferred.

In a further preferred embodiment, the effect of the agent according to the present invention can be enhanced by addition of emulsifiers. Such emulsifiers include addition products of ethylene oxide and/or propylene oxide with linear fatty alcohols, with fatty acids, and with alkylphenols; C₁₂ to C₂₂ fatty acid mono- and diesters of addition products of ethylene oxide with polyols, in particular with glycerol; C₈ to C₂₂ alkyl mono- and oligoglucosides and ethoxylated analogs thereof; addition products of ethylene oxide with castor oil and hardened castor oil; sterols; phospholipids; fatty acid esters of sugars and sugar alcohols such as sorbitol; polyglycerols and polyglycerol derivatives such as polyglycerol poly-12-hydroxystearate, as well as linear and branched fatty acids having 8 to 30 carbon atoms, and physiologically acceptable salts thereof. Agents according to the present invention contain emulsifiers preferably in quantities from 0.1 to 25 wt %, particularly 0.5 to 15 wt %, based on total weight of the ready-to-use agent.

Nonionogenic emulsifiers or surfactants having an HLB value from 10 to 15 can be particularly preferred according to the present invention.

Preferred ready-to-use agents possess a pH from 7.0 to 12.0, preferably from 7.5 to 11.5, particularly preferably from 8.0 to 11.0. Acidifying and alkalizing agents familiar to one skilled in the art of cosmetics are commonly used to adjust pH. These alkalizing agents are typically chosen from inorganic salts, particularly the alkali and alkaline-earth metals, organic alkalizing agents, in particular amines, basic amino acids and alkanolamines, and ammonia. Useful organic alkalizing agents are preferably chosen from alkanolamines made up of primary, secondary, or tertiary amines having a C₂ to C₆ alkyl base element that carries at least one hydroxyl group. Particularly preferred alkanolamines are monoethanolamine and triethanolamine. Basic amino acids usable as an alkalizing agent according to the present invention are preferably chosen from arginine and lysine. Preferred inorganic alkalizing agents are chosen from sodium hydroxide, potassium hydroxide, calcium hydroxide, barium hydroxide, sodium phosphate, potassium phosphate, sodium silicate, potassium silicate, sodium carbonate, and potassium carbonate. Sodium hydroxide and/or potassium hydroxide are particularly preferred. Lastly, a further preferred alkalizing agent is ammonia. Acidifying agents preferred according to the present invention include edible acids such as citric acid, acetic acid, malic acid, or tartaric acid, as well as dilute mineral acids.

The pH values are, for purposes of the present invention, pH values that have been measured at a temperature of 22° C. The pH adjusting agents are preferably present in an amount of from 0.05 to 10 wt %, particularly from 0.5 to 5 wt %, based on total weight of the ready-to-use agent.

Agents according to the present invention can moreover contain further active substances, adjuvants, and additives, among them:

nonionic polymers such as vinylpyrrolidinone/vinyl acrylate copolymers, polyvinylpyrrolidinone, vinylpyrrolidinone/vinyl acetate copolymers, polyethylene glycols, and polysiloxanes;
cationic polymers such as quaternized cellulose ethers, polysiloxanes having quaternary groups, dimethyldiallylammonium chloride polymers, acrylamide/dimethyldiallylammonium chloride copolymers, dimethylaminoethyl methacrylate/vinylpyrrolidinone copolymers quaternized with diethyl sulfate, vinylpyrrolidinone/imidazolinium methochloride copolymers, and quaternized polyvinyl alcohol;
zwitterionic and amphoteric polymers such as acrylamidopropyltrimethylammonium chloride/acrylate copolymers and octylacrylamide/methyl methacrylate/t-butylaminoethyl methacrylate/2-hydroxypropyl methacrylate copolymers;
anionic polymers such as polyacrylic acids, crosslinked polyacrylic acids, vinyl acetate/crotonic acid copolymers, vinylpyrrolidinone/vinyl acrylate copolymers, vinyl acetate/butyl maleate/isobornyl acrylate copolymers, methylvinyl ether/maleic acid anhydride copolymers, and acrylic acid/ethyl acrylate/N-t-butylacrylamide terpolymers;
further thickening agents such as agar-agar, guar gum, alginates, gum arabic, karaya gum, locust bean flour, linseed gums, dextrans, cellulose derivatives (e.g., methyl cellulose, hydroxyalkyl cellulose), and carboxymethyl cellulose, starch fractions and derivatives such as amylose, amylopectin, and dextrins, or entirely synthetic hydrocolloids such as polyvinyl alcohol; hair-conditioning compounds such as phospholipids, in particular lecithins and kephalins, as well as silicone oils;
protein hydrolysates such as hydrolysates of elastin, collagen, keratin, milk protein, soy protein, and wheat protein, condensation products thereof with fatty acids, and quatemized protein hydrolysates;
organic solvents such as ethylene glycol, propylene glycol, and diethylene glycol; fiber-structure-improving active substances, in particular mono-, di- and oligosaccharides such as glucose, galactose, fructose, fruit sugars, and lactose;
structuring agents such as maleic acid and lactic acid;
defoamers such as silicones, preferably dimethicone;
dyes for coloring the agent; anti-dandruff active substances such as zinc omadine and climbazol;
light-protection agents and UV blockers, in particular derivatized benzophenones, cinnamic acid derivatives, and triazines;
active substances such as panthenol, pantothenic acid, pantolactone, allantoin, pyrrolidinonecarboxylic acids, and salts thereof, as well as bisabolol; vitamins, provitamins, and vitamin precursors, in particular those of the groups A, B₃, B₅, B₆, C, E, F, and H; cholesterol;
consistency agents such as sugar esters, polyol esters, or polyolalkyl ethers; oils and other fatty substances, such as vegetable or synthetic oils, usually triglyceride-based, and fatty substances such as fatty alcohols as well as esters thereof with fatty acids;
fats and waxes such as beeswax, Montan wax, and paraffins;
swelling and penetration substances such as glycerol, propylene glycol monoethyl ether, carbonates, hydrogencarbonates, guanidines, ureas, and primary, secondary, and tertiary phosphates;
opacifiers such as latex, styrene/PVP and styrene/acrylamide copolymers; luster agents such as ethylene glycol mono- and distearate as well as PEG-3 distearate;
pigments;
perfume oils;
antioxidants; and
propellants such as propane/butane mixtures, N₂O, dimethyl ether, CO₂, and air.

One skilled in the art selects these further substances according to the desired properties of the agents. Regarding further optional components, as well as the quantities of those components used, reference is made to relevant manuals known to one skilled in the art (e.g., K h. Schrader, Grundlagen and Rezepturen der Kosmetika [Cosmetics fundamentals and formulations], 2nd ed., Hüthig Buch Verlag, Heidelberg (1989)).

Agents according to the present invention are particularly suitable for achieving uniform and homogeneous lightening.

A further subject of the present invention is therefore the use of an agent containing in a cosmetic carrier at least one oxidizing agent, preferably hydrogen peroxide, and furthermore containing at least one particulate component for homogeneous lightening of keratinic fibers, particularly human hair. The oxidizing agent is preferably hydrogen peroxide and the particulate constituent comprises a particle core preferably chosen from solid alkalizing agents and/or solid peroxo compounds and having a casing surrounding this core, wherein the casing contains as an encapsulating material at least shellac, preferably a mixture of shellac and talc.

Statements made concerning the agents and coated particles according to the present invention apply mutatis mutandis, in terms of material selection with respect to preferred embodiments of the use according to the present invention.

In order to prevent premature, undesired release and/or reaction of the coated particles having the oxidizing agent, the encased particles and oxidizing agent can be packaged separately from one another and brought into contact with one another by mixing only immediately before utilization.

Coated particles can be made available in a variety of presentation forms, for example, as pastes, powders, tablets, etc., provided they contain at least one particulate ingredient that is surrounded by a casing. In terms of utilization convenience, however, agents according to the present invention are particularly present as hair-bleaching powders. In a preferred embodiment, the coated particles are present in the form of a powder or a fine granulate that optionally also contains further non-encased components.

In this embodiment, the powder or granulate containing the coated particles—hair-bleaching powder (A)—is provided in a separate container, preferably in the form of a small bag, a sachet, or a pouch, or also in the form of an (optionally reclosable) can.

In a further embodiment of the present invention, agents which are manufactured immediately before use by mixing at least two preparations are therefore preferred. Agents of this embodiment are preferably marketed as multi-component package units (kit of parts).

A further embodiment of the present invention is therefore a multi-component package unit (kit of parts) for lightening keratinic fibers, wherein it comprises at least two containers packaged separately from one another, one container having a preferably powdered hair-bleaching preparation (A) containing at least coated particles according to the first subject of the invention, and a further container having an oxidizing agent composition (B) containing at least one chemical oxidizing agent, in particular hydrogen peroxide.

When the hair-bleaching preparation (A) is present in the form of a powder or fine granulate, it may be advantageous, in order to manufacture the ready-to-use lightening agent, to first mix the hair-bleaching preparation (A) with an additional liquid cream or emulsion base (C), and then add an oxidizing agent preparation (B).

The cream or emulsion base (C) is a common cosmetic base, usually water-based, and contains surfactants, emulsifiers, fatty substances, and thickeners that are common in cosmetics, as well as further active substances and adjuvants that one skilled in the art will assemble as necessary.

A further embodiment of the present invention is therefore a multi-component package unit (kit of parts) for lightening keratinic fibers, wherein it comprises at least three containers packaged separately from one another, one container (I) containing a hair-bleaching preparation (A), a second container (III) a preparation (C), and a further container (II) an oxidizing agent preparation (B) containing at least one oxidizing agent.

The multi-component package unit (kit of parts) preferably also contains a set of instructions that explains the utilization sequence, as well as optionally the mixing sequence for the individual preparations. It may also be preferred if an application aid, for example, a comb or a brush, and/or personal protection equipment such as disposable gloves are added to the kit.

Statements made concerning agents and coated particles according to the present invention apply mutatis mutandis with respect to further embodiments of the multi-component package units.

A further subject of the present invention is a method for manufacturing agents for lightening keratin fibers comprising the steps of:
a) applying a solution or dispersion of a coating agent containing at least shellac and, optionally, additionally talc, to at least one particulate constituent, thereby forming a casing, and then
b) mixing the coated particle(s) with an oxidizing preparation containing hydrogen peroxide.

In the first step of the method according to the present invention, at least one particulate ingredient is coated with at least shellac. This step can be carried out without difficulty in a wide variety of equipment.

Casing materials can be sprayed as an aqueous dispersion or dispersion using fluidized bed technology (using the bottom spray method with or without the Wurster process, or the tangential spray method) onto the particulate ingredients, which are dried at the same time in the air flow so that a uniform, almost 100% layer encloses the powder particles. Methods according to the present invention in which the coating of the particles is carried out in a fluidized bed apparatus are preferred.

In the event of insufficient coating using the manufacturing technology described above (fluidized bed coating), coatings with the film materials recited above are alternatively possible using spouted bed technology. A further possibility for encasing powder particles includes melt extrusion technology and use of a mixer granulator.

In order to prevent sticking or caking of the encased particles, it has proven to be particularly effective if the particles contain silicon dioxide on the surface as an additional separating agent. For this purpose, particles which have been encased with one or more encapsulation layers (that may optionally differ in thickness and composition) are sprayed with an aqueous dispersion of silicon dioxide and dried.

Coating the particle cores according to step a) is thus preferably carried out in two stages, wherein the particle cores to be coated, in a fluidized bed apparatus, are in a first stage, sprayed with a dispersion containing at least shellac as well as optionally talc and/or additional casing materials and adjuvants and dried, and in a second stage, sprayed with an aqueous dispersion containing silicon dioxide and dried.

Encased particles obtained thereby serve for manufacture of the ready-to-use lightening agent by mixing the coated particle(s) with an oxidizing preparation containing hydrogen peroxide.

A preferred embodiment of the method according to the present invention is wherein the particulate constituents encased in accordance with step a) are, after that step, additionally sprayed with an aqueous dispersion containing silicon dioxide, and then dried before mixing step b) occurs.

Statements made concerning the agents and coated particles according to the present invention apply mutatis mutandis with respect to further embodiments of the method according to the present invention.

We claim:

1. Coated particles comprising:
a solid particle core, and
a casing surrounding the core,
wherein the casing comprises shellac.

2. Coated particles according to claim 1, wherein the casing further comprises talc.

3. Coated particles according to claim 1, wherein the particle core comprises solid alkalizing agents and/or solid peroxo compounds.

4. Coated particles according to claim 3, wherein the particle core is at least a solid alkalizing agent chosen from alkali metal carbonates, metasilicates, silicates, and phosphates.

5. Coated particles according to claim 3, wherein the particle core is at least a solid peroxo compound chosen from hydrogen peroxide addition compounds on solid carriers, ammonium peroxomonosulfates, alkali metal peroxomonosulfates, ammonium peroxodisulfates, and alkali metal peroxodisulfates.

6. Coated particles according to claim 2, wherein the shellac and talc are present in the casing in an amount of at least 50 wt %, based on total weight of the casing.

7. Coated particles according to claim 2, wherein the shellac and talc are present in the casing at a weight ratio to one another of from 5:1 to 1:1.

8. Coated particles according to claim 1, wherein the casing further comprises a cellulose derivative.

9. Coated particles according to claim 8, wherein the cellulose derivative is chosen from hydroxypropylmethyl cellulose and/or hydroxypropyl cellulose.

10. Coated particles according to claim 1, wherein the casing further comprises a disintegration agent.

11. Coated particles according to claim 1, wherein the particle core further comprises silicon dioxide as an additional separating agent.

12. Agent for lightening keratinic fibers comprising, in a cosmetic carrier, at least one oxidizing agent and coated particles according to claim 1.

13. Agent according to claim 12 wherein the oxidizing agent is at least hydrogen peroxide.

14. Multi-component package unit (kit of parts) for lightening keratinic fibers comprising at least two containers packaged separately from one another, one container comprising a hair-bleaching preparation having coated particles according to claim 1, and a further container comprising an oxidizing agent composition having at least one chemical oxidizing agent.

15. Multi-component package according to claim 14 wherein the hair-bleaching preparation is in the form of a powder.

16. Multi-component package according to claim 14 wherein the chemical oxidizing agent is at least hydrogen peroxide.

17. A method for manufacturing agents for lightening keratin fibers, comprising the steps of:
a) applying to at least one particulate constituent a solution or dispersion of a coating agent comprising shellac and optionally talc,
b) forming a casing over the particulate constituent, and
c) mixing the coated particle(s) with an oxidizing preparation containing hydrogen peroxide.

18. Method according to claim 17, wherein the particulate constituents encased in accordance with step b) are, after that step, additionally sprayed with an aqueous dispersion containing silicon dioxide, and then drying the particulate constituents before mixing step c) occurs.
